(19) United States
(12) Patent Application Publication
(10) Pub. No.: US 2021/0282671 A1
(43) Pub. Date: Sep. 16, 2021

(54) HUMIDITY-BASED SWEAT RATE SENSING DEVICES

(71) Applicant: Eccrine Systems, Inc., Cincinnati, OH (US)

(72) Inventors: Michelle Hoffman, Wyoming, OH (US); Jason Heikenfeld, Cincinnati, OH (US); Jacob A Bertrand, Montgomery, OH (US); Nicholas Bailey, Cincinnati, OH (US)

(73) Assignee: Eccrine Systems, Inc., Cincinnati, OH (US)

(21) Appl. No.: 16/605,979

(22) PCT Filed: Mar. 22, 2019
(86) PCT No.: PCT/US19/23665

§ 371 Date: Oct. 17, 2019

Related U.S. Application Data
(60) Provisional application No. 62/647,013, filed on Mar. 23, 2018.

(51) Int. Cl. A61B 5/145; A61B 10/00; A61B 5/01

(52) U.S. Cl. CPC A61B 5/14517; A61B 2562/029; A61B 5/01; A61B 10/0064

(57) ABSTRACT

Embodiments of the disclosed invention provide wearable devices that use a humidity sensor to measure sweat rate generated from an area of skin. A sensing chamber is continuously filled with a sweat sample, which forms a droplet and alters the humidity measured within the chamber. Once the sweat sample droplet expands to the edge of the chamber, the droplet contacts a wick and is drawn away, so the chamber can fill with a subsequent droplet. The device uses a droplet volume and the time required to reach a maximum humidity to calculate a sweat rate. A pump is used to draw old sweat sample out of the wick to allow extended device operation. Some embodiments also include capacitive sensors to perform back up measurements. Another set of embodiments includes alternatively shaped sensing chambers configured to reduce sample volumes or improve function. A method for determining sweat rate based on humidity sensor measurements is also included.

### CROSS-REFERENCE TO RELATED APPLICATIONS

This application claims priority to PCT/US19/23665, filed Mar. 22, 2019, and U.S. Provisional Application No. 62/647,013, filed Mar. 23, 2018; and has specification that builds upon PCT/US16/36038, filed Jun. 6, 2016; PCT/US17/42677 and U.S. Ser. No. 15/653,494, filed Jul. 18, 2017; and PCT/US2018/52176, filed Sep. 21, 2018, the disclosures of which are hereby incorporated by reference herein in their entirety.

### BACKGROUND OF THE INVENTION

Sweat contains many of the same biomarkers, chemicals, or solutes that are carried in blood and can provide significant information enabling one to diagnose illness, health status, exposure to toxins, performance, and other physiological attributes even in advance of any physical sign. Furthermore, sweat itself, the action of sweating, and other parameters, attributes, solutes, or features on, near, or beneath the skin can be measured to further reveal physiological information. Of the other physiological fluids used for bio monitoring (e.g., blood, urine, saliva, tears), sweat has arguably the least predictable sampling rate in the absence of technology. However, with proper application of technology, sweat can potentially outperform other non-invasive or less invasive biofluids in predictable sampling.

Despite its potential, the state of art in biological monitoring through sweat sensing is in need of additional devices and methods to properly determine sweat rates. An accurate measure of sweat rate may prove critical for a number of sweat sensing device applications, including the determination of total body fluid loss, determination of analyte concentrations in sweat, establishment of chronologically assured sampling rates, estimates of sweat sensing device operational lifespan, or determining a degree of sweat concentration with respect to a sweat analyte, among others.

While techniques for measuring sweat rate are known in the art, such as the volumetric sweat rate devices disclosed in PCT/US17/42677 and U.S. Ser. No. 15/653,494, which are hereby incorporated by reference in their entirety, such approaches face challenges due to fluid flow continuity. For example, a volumetric sweat rate sensor employs a channel with defined volume that receives a biofluid sample from the skin. The device measures time required for the fluid front to engage pairs of co-planar electrodes, and then uses the channel volume between electrode pairs to generate a flow rate. Such measurements are vulnerable to interruptions in fluid flow, since the sweat sample must remain cohered as it wets into the channel and engages with the electrode pairs. This requirement is particularly challenging in a wearable system subject to acceleration related to body movement, which can generate enough force to break the front of the fluid. As a result, multiple failure modes are possible, including missed electrodes, which cause reduced temporal resolution; false-positive electrode engagement, which overestimates flow rates; and wicking along the channel wall, which can corrupt all device measurements.

Another sweat rate sensing modality, as disclosed in PCT/US2018/52176 measures discrete drops of sweat as they are attracted by a current and wicked away. The disclosed invention improves upon such devices by providing a simplified sensing modality, and in some embodiments, redundancy though the use of multiple modalities. In addition, at least one humidity-based sweat rate sensor is also known in the art, see, U.S. Pat. No. 5,131,390, Sakaguchi, et al., however, this device is complex, requires the use of dehumidified air, and does little to translate measurements into physiologically meaningful information.

What is needed, therefore, are alternative devices and methods that can provide reliable determination of sweat rate in a simple, wearable device.

### SUMMARY OF THE INVENTION

Embodiments of the disclosed invention provide wearable devices that use a humidity sensor to measure sweat rate generated from an area of skin. A sensing chamber is continuously filled with a sweat sample, which forms a droplet and alters the humidity measured within the chamber. Once the sweat sample droplet expands to the edge of the chamber, the droplet contacts a wick and is drawn away, so the chamber can fill with a subsequent droplet. The device uses a droplet volume and the time required to reach a maximum humidity to calculate a sweat rate. A pump is used to draw old sweat sample out of the wick to allow extended device operation. Some embodiments also include capacitive sensors to perform back up measurements. Another set of embodiments includes alternatively shaped sensing chambers configured to reduce sample volumes or improve function. A method for determining sweat rate based on humidity sensor measurements is also included.

### BRIEF DESCRIPTION OF THE DRAWINGS

The disclosed invention will be further appreciated in light of the following descriptions and drawings in which:

FIG. 1 depicts at least a portion of a sweat sensing device comprising the disclosed invention.

FIG. 2 depicts a cross-sectional view of at least a portion of a device comprising the disclosed invention.

FIG. 2A depicts a top-down view of the device depicted in FIG. 2 as if bisected along the line 20.

FIG. 3 depicts a cross-sectional view of at least a portion of a device comprising the disclosed invention.

FIG. 4 depicts a top-down view of at least a portion of a device comprising the disclosed invention.

FIG. 4A depicts an alternate embodiment of the device depicted in FIG. 4.

FIG. 4B depicts a radial cross-sectional view of the device depicted in FIG. 4 as if divided along the line 22.

FIG. 5 depicts a cross-sectional view of at least a portion of a device comprising the disclosed invention.

FIGS. 5A and 5B depict alternate embodiments of the device depicted in FIG. 5.

### DEFINITIONS

As used herein, “sweat” means a biofluid that is primarily sweat, such as eccrine or apocrine sweat, and may also include mixtures of biofluids such as sweat and blood, or sweat and interstitial fluid, so long as advective transport of the biofluid mixtures (e.g., flow) is primarily driven by sweat.

“Sweat sensor” means any type of sensor that measures a state, presence, flow rate, solute concentration, or solute presence, in absolute, relative, trending, or other ways in sweat. Sweat sensors can include, for example, potentiometric, amperometric, impedance, optical, mechanical, antibody, peptide, aptamer, or other means known by those skilled in the art of sensing or biosensing.

“Analyte” means a substance, molecule, ion, or other material that is measured by a sweat sensing device.

“Measured” can imply an exact or precise quantitative measurement and can include broader meanings such as, for example, measuring a relative amount of change of something. Measured can also imply a qualitative measurement, such as ‘yes’ or ‘no’ type measurements.

“Chronological assurance” means the sampling rate or sampling interval that assures measurement(s) of analytes in sweat in terms of the rate at which measurements can be made of new sweat analytes emerging from the body. Chronological assurance may also include a determination of the effect of sensor function, potential contamination with previously generated analytes, other fluids, or other measurement contamination sources for the measurement(s). Chronological assurance may have an offset for time delays in the body (e.g., a well-known 5 to 30-minute lag time between analytes in blood emerging in interstitial fluid), but the resulting sampling interval (defined below) is independent of lag time, and furthermore, this lag time is inside the body, and therefore, for chronological assurance as defined above and interpreted herein, this lag time does not apply.

“Analyte-specific sensor” means a sensor specific to an analyte and performs specific chemical recognition of the analyte's presence or concentration (e.g., ion-selective electrodes (“ISE”), enzymatic sensors, electro-chemical aptamer based sensors, etc.). Sensors could also be optical, mechanical, or use other physical/chemical methods which are specific to a single analyte. Further, multiple sensors can each be specific to one of multiple analytes.

“Sweat sensor data” means all of the information collected by sweat system sensor(s) and communicated via the system to a user or a data aggregation location.

“Correlated aggregated sweat sensor data” means sweat sensor data that has been collected in a data aggregation location and correlated with outside information such as time, temperature, weather, location, user profile, other sweat sensor data, or any other relevant data.

“Sweat sampling rate” means the effective rate at which new sweat, or sweat solutes, originating from the sweat gland or from skin or tissue, reaches a sensor that measures a property of sweat or its solutes. Sweat sampling rate, in some cases, can be far more complex than just sweat generation rate. Sweat sampling rate directly determines, or is a contributing factor in determining chronological assurance. Times and rates are inversely proportional (rates having at least partial units of 1/seconds), therefore a short or small time required to refill a sweat volume can also be said to have a fast or high sweat sampling rate. The inverse of sweat sampling rate (1/s) could also be interpreted as a “sweat sampling interval”. Sweat sampling rates or intervals are not necessarily regular, discrete, periodic, discontinuous, or subject to other limitations. Like chronological assurance, sweat sampling rate may also include a determination of the effect of potential contamination with previously generated sweat, previously generated solutes, other fluid, or other measurement contamination sources for the measurement(s). Sweat sampling rate can also be in whole or in part determined from solute generation, transport, advective transport of fluid, diffusion transport of solutes, or other factors that will impact the rate at which new sweat or sweat solutes reach a sensor and/or are altered by older sweat or solutes or other contamination sources. Sensor response times may also affect sampling rate.

“Sweat generation rate” or “sweat rate” means the rate at which sweat is generated by the sweat glands themselves. Sweat generation rate is typically measured by the flow rate from each gland in nL/min/gland. In some cases, the measurement is then multiplied by the number of sweat glands from which the sweat is being sampled. For example, assuming 100 active glands/cm², if a sweat collector covered an area of 1 cm² and detected 100 nL of sweat per minute, the device would determine a sweat rate of 1 nL/min/gland, and 100 nL/min/cm², both of which can be extrapolated to a total body sweat rate.

“Measured” may mean an exact or precise quantitative measurement and can include broader meanings such as, for example, measuring a relative amount of change of something. Measured can also mean a binary measurement, such as ‘yes’ or ‘no’ type measurements.

“Sweat volume” means the fluidic volume in a space that can be defined multiple ways. Sweat volume may be the volume that exists between a sensor and the point of generation of sweat, or between a sensor and a solute moving into or out of sweat from the body or from other sources. Sweat volume can include the volume that can be occupied by sweat between the sampling site on the skin and a sensor on the skin, where the sensor has no intervening layers, materials, or components between it and the skin; or between the sampling site on the skin and a sensor on the skin where there are one or more layers, materials, or components between the sensor and the sampling site on the skin. Sweat volume may refer to the sweat volume of multiple integrated components, or used in description of the sweat volume for single component or a subcomponent, or in the space between a device, or device component, and skin.

“Microfluidic components” means channels in polymer, textiles, paper, or other components known in the art of microfluidics for guiding movement of a fluid or at least partial containment of a fluid. This has served as a background for the present invention, including background technical invention needed to fully appreciate the present invention, which will now be summarized.

### DETAILED DESCRIPTION OF THE INVENTION

The disclosed invention includes a novel design for a humidity sensor-based sweat rate sensor which is not reliant on the generation of bulk fluid flow.

To clarify the proper numerical values or representations of sweat sampling rate and therefore chronological assurance, sweat generation rate and sweat volumes will be described in detail. From Dermatology: an illustrated color text, 5th ed., the maximum sweat generated per person per day is 10 L, which on average is 4 μL, per gland maximum per day, or about 3 nL/min/gland. This is about 20× higher than the minimum sweat generation rate. The maximum stimulated sweat generation rate according to Buono 1992, J. Derm. Sci. 4, 33-37, “Cholinergic sensitivity of the eccrine sweat gland in trained and untrained men,” the maximum sweat generation rate by pilocarpine stimulation is about 4 nL/min/gland for untrained men and 8 nL/min/gland for trained (exercising often) men. Sweat stimulation data from “Pharmacologic responsiveness of isolated single eccrine sweat glands,” by K. Sato and F. Sato, Am. Physiological Society, Jul. 30, 1980, suggests a sweat generation rate up to about 5 nL/min/gland is possible with stimulation, and several types of sweat stimulating substances are disclosed (the data was for extracted and isolated monkey sweat glands, which are very similar to human ones). For simplicity, we can assume for calculations in the present disclosure (without so limiting the disclosure), that the minimum sweat generation rate is about 0.1 nL/min/gland, and the maximum sweat generation rate is about 5 nL/min/gland, which is about a 50× difference between the maximum and minimum rates.

Based on the assumption of a sweat gland density of 100/cm², a sensor that is 0.55 cm in radius (1.1 cm in diameter) would cover about 1 cm² area, or approximately 100 sweat glands. Next, assume a sweat volume under a skin-facing sensor (space between the sensor and the skin) of 100 μm average height or 100E-4 cm, and that same 1 cm² area, which provides a sweat volume of 100E-4 cm³ or about 100E-4 mL or 10 μL of volume. With the maximum sweat generation rate of 5 nL/min/gland and 100 glands, it would require a 20 minutes to fully refresh the sweat volume (using first principles/simplest calculation only). With the minimum sweat generation rate of 0.1 nL/min/gland and 100 glands, it would require 1000 minutes or ˜17 hours to refresh the sweat volume. Because the flow is not entirely centered, according to Sonner, et al., in Biomicrofluidics, 2015 May 15; 9(3):031301. doi: 10.1063/1.4921039, the time to fully refresh the sweat volume (i.e., new sweat replaces all old sweat) could be 6× longer or more. For slow sweat flow rates, back-diffusion of analytes and other confounding factors could make the effective sampling interval even larger. Clearly, conventional wearable sweat sensing approaches with large sweat volumes and slow sampling rates would find continuous sweat sample monitoring to be a significant challenge.

Sweat stimulation, or sweat activation, can be achieved by known methods. For example, sweat stimulation can be achieved by simple thermal stimulation, chemical heating pad, infrared light, by orally administering a drug, by intradermal injection of drugs such as carbachol, methylcholine or pilocarpine, and by dermal introduction of such drugs using iontophoresis, by sudo-motor-axon reflex sweating, or by other means. A device for iontophoresis may, for example, provide direct current and use large lead electrodes lined with porous material, where the positive pole is dampened with 2% pilocarpine hydrochloride or carbachol and the negative one with 0.9% NaCl solution. Sweat can also be controlled or created by asking the device wearer to conduct or increase activities or conditions that cause them to sweat.

One skilled in the art will recognize that the various embodiments may be practiced without one or more of the specific details described herein, or with other replacement and/or additional methods, materials, or components. In other instances, well-known structures, materials, or operations are not shown or described in detail herein to avoid obscuring aspects of various embodiments of the invention. Similarly, for purposes of explanation, specific numbers, materials, and configurations are set forth herein in order to provide a thorough understanding of the invention. Furthermore, it is understood that the various embodiments shown in the figures are illustrative representations and are not necessarily drawn to scale.

Reference throughout this specification to “one embodiment” or “an embodiment” means that a particular feature, structure, material, or characteristic described in connection with the embodiment is included in at least one embodiment of the invention, but does not denote that they are present in every embodiment. Thus, the appearances of the phrases “in an embodiment” or “in another embodiment” in various places throughout this specification are not necessarily referring to the same embodiment of the invention. Further, “a component” may be representative of one or more components and, thus, may be used herein to mean “at least one.”

Certain embodiments of the invention show sensors as simple individual components. It is understood that many sensors require two or more electrodes, reference electrodes, or additional supporting technology or features that are not captured in the description herein. Sensors are preferably electrical in nature, but may also include optical, chemical, mechanical, or other known biosensing mechanisms. Sensors can be in duplicate, triplicate, or more, to provide improved data and readings. Sensors may be referred to by what the sensor is sensing, for example: a sweat sensor; an impedance sensor; a fluid volume sensor; a sweat generation rate sensor; and a solute generation rate sensor. Certain embodiments of the disclosed invention show sub-components of what would be fluid sensing devices with more sub-components needed for use of the device in various applications, which are obvious (such as a battery), and for purpose of brevity and focus on inventive aspects are not explicitly shown in the diagrams or described in the embodiments of the invention. As a further example, many embodiments of the invention could benefit from mechanical or other means known to those skilled in wearable devices, patches, bandages, and other technologies or materials affixed to skin, to keep the devices or sub-components of the skin firmly affixed to skin or with pressure favoring constant contact with skin or conformal contact with even ridges or grooves in skin, and are included within the spirit of the disclosed invention.

The disclosed sweat sensing device also includes computing and data storage capability sufficient to operate the device, which incorporates the ability to conduct communication among system components, to perform data aggregation, and to execute algorithms capable of generating notification messages. The device may have varying degrees of onboard computing capability (i.e., processing and data storage capacity). For example, all computing resources could be located onboard the device, or some computing resources could be located on a disposable portion of the device and additional processing capability located on a reusable portion of the device. Alternatively, the device may rely on portable, fixed or cloud-based computing resources.

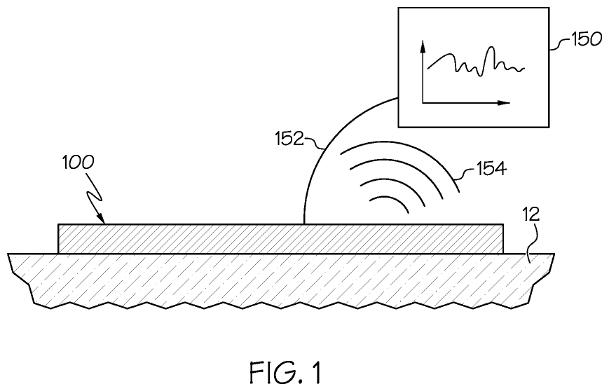

With reference to FIG. 1, a sweat sensing device 100 is placed on or near skin 12. In an alternate embodiment, the sweat sensor device may be simply fluidically connected to skin or regions near skin through microfluidics or other suitable techniques. The device 100 is in wired communication 152 or wireless communication 154 with a reader device 150. In some embodiments, reader device 150 may be a smart phone or portable electronic device. In alternate embodiments, device 100 and reader device 150 can be combined. In further alternate embodiments, communication 152 or 154 is not constant and could be a periodic or one-time data transmission from device 100 once it has completed its measurements of sweat. In various embodiments, the device 100 may be reusable, disposable, or may combine reusable and disposable components. For example, electronics and communications components may be reusable, while wicking components and sensors may be disposable. In particular, humidity sensors can achieve useful lifespans up to several months absent fouling by excess fluid or humidity, and therefore may be included in a reusable component. Multiple combinations are possible and contemplated herein.

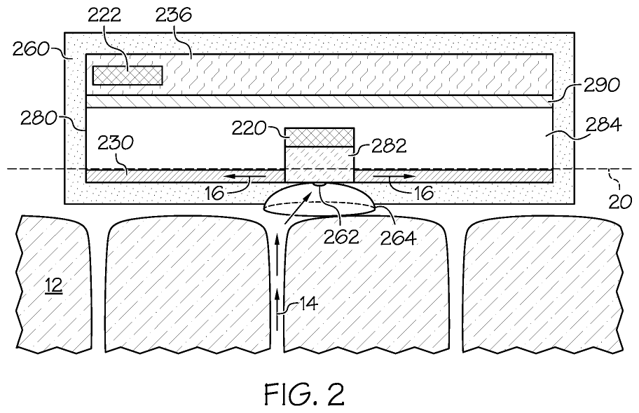

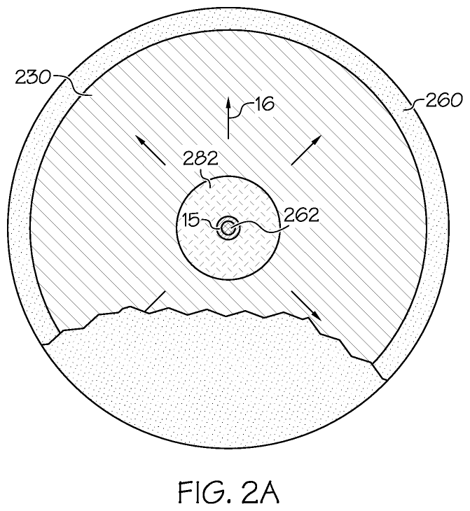

FIG. 2 depicts a sweat sensing device employing a humidity-based sweat rate sensor of the disclosed invention. The sweat sensing device is shown being worn on an individual's skin 12. The device includes a fluid impermeable substrate or case 260, made from, e.g., PET, or PVC. The case creates an enclosure 280 having a volume of, e.g., 5 mL, and has an inlet 262 through the skin-facing side of the case. The inlet may be located within a sweat collector 264, the sweat collector including a concave surface facing the skin, a substantially circular seal, and optionally a plurality of ridges extending radially from the inlet toward the seal to facilitate spacing between the skin and the inlet. The sweat collector creates a defined collection area on the skin, e.g., 1 cm², and allows the skin to bulge into the collector to create a seal, while preventing occlusion of sweat ducts. Some embodiments may create or augment the seal by using adhesives, o-rings, hydrophobic coatings, or other suitable means to prevent sweat exiting the collector except via the inlet, or entering the collection area from skin outside the seal. The sweat collector as described therefore prevents contamination of the sweat sample from outside the seal, retains sweat generated within the collection area, and promotes measurement of a physiologically relevant sweat rate that can be extrapolated to whole-body sweat loss or other relevant information. Alternatively, the device may use a wicking collector (not shown), such as those described in U.S. Ser. No. 16/206,095, filed Nov. 30, 2018 and incorporated herein by reference in its entirety. In such embodiments, instead of a concave sweat collector 264 and vertical inlet 262, the device would convey a sweat sample into the device by the wicking collector, where the sweat sample would interact with the device sensors.

In fluidic communication with the inlet 262 is a humidity sensing chamber 282. Within the humidity sensing chamber 282 is one or more humidity sensors 220 (one is shown). The humidity sensing chamber is adjacent to, or surrounded by a wick 230, see FIG. 2A, which depicts a top-down view of the device of FIG. 2 as if sliced along the dashed line 20. The wick is in fluidic communication with the humidity sensing chamber 282 and a humidity dissipation volume 284, and may be constructed of nylon, a polymer, paper, textile, rayon, or other suitable material for transporting the biofluid sample out of, and away from, the humidity sensing chamber. The humidity sensing chamber has a known volume, e.g., 1 mL, and with the exception of the inlet 262 and wick 230, is sealed from the outside environment. While a cylindrical volume is depicted, different geometries are possible for the sensing chamber. In particular, the height of the sensing chamber (the axis extending from the inlet 262 to the sensor 220) may be shortened to provide a lower overall device profile, as long as the volume of the sensing chamber is adequate for the application.

A droplet formation area, meaning the floor of the sensing chamber surrounding the inlet, may be coated with various materials known in the art of microfluidics to achieve desired flow results. For example, the chamber surface may have a hydrophobic coating that promotes the formation of a sweat sample droplet within the sensing chamber. As a result, the droplet could form in a substantially spherical shape before reaching the wick and being removed. Wetting of the droplet formation area should be avoided so that capillary flow is not established to wick the sweat sample droplet out of the sensing chamber prematurely. The droplet formation area must be large enough to allow a droplet of sufficient size to form and persist in the sensing chamber long enough to be accurately measured by the humidity sensor. This area will depend on the saturation vapor pressure of water (as determined by an Arcien Buck equation,

$${P}_{s} = 0.61121 ( ( 18.678 \frac{T}{234.5} ) ( \frac{T}{257.14 + T} ) ) ) ,$$

and the capabilities of the humidity sensor, e.g., the time and % humidity resolutions of the sensor.

A boundary of the dissipation volume 284 is created by a membrane 290. The membrane may be, e.g., a selectively permeable membrane, a vapor porous membrane, an osmosis membrane, a dialysis membrane, a track-etch membrane, or other suitable material that allows the passage of moisture out of the dissipation volume. The membrane spans a dimension of the enclosure 280, and isolates a portion of the enclosure to comprise a pump 236. The pump also includes an absorbent material, e.g., a desiccant, paper, an absorbent hydrogel, or other material suitable for drawing biofluid out of the wick 230 and/or the dissipation volume 284. Some embodiments also include a pump humidity sensor 222 within the pump. Some embodiments may include one or more analyte-specific sensors (not shown), e.g., ion-selective electrode sensors, electrochemical aptamer based sensors, amperometric, or enzymatic sensors. Other embodiments include one or more secondary sensors (not shown), which may be, e.g., a temperature sensor, a volumetric sweat rate sensor, a micro-thermal flow rate sensor, a discrete droplet volume system (as disclosed in PCT/US2018/52176), a galvanic skin response sensor (GSR), a sweat conductivity sensor, or impedance or capacitance sensors for skin contact measurement.

In operation, the disclosed sweat rate measurement device will receive a sweat sample from the skin 12 that moves generally as depicted by the arrow 14, and passes through the inlet 262 and into the humidity sensing chamber 282. The humidity sensing chamber has a fixed volume, which at the beginning of a sampling cycle is filled with air and ambient water vapor, and substantially devoid of sweat. Humidity sensors typically report relative humidity, which is governed by the equation

$$R . H . = ( \frac{E}{{E}_{S}} ) * 100 \% ,$$

where E is the actual vapor density or measured humidity

$$( E = \frac{- {m}_{{H}_{2} O}}{V} ,$$

where m_{H}_{₂}_{O} is the mass of water vapor, and V is the volume of the air/water vapor mix), and Eₛ is the saturation vapor density, which depends on temperature. When the chamber is empty, the humidity sensor will measure a baseline value. As a sweat sample fills into the chamber, a portion of the chamber volume fills with sweat, and the volume of air in the chamber decreases. As a result, the humidity sensor will register an increase in R.H. that is directly correlated to the volume of the sweat sample in the chamber. While filling the sensing chamber, a roughly spherical sweat sample droplet will increase in size both vertically and horizontally within the droplet formation area. With further reference to FIG. 2A, when the horizontal expansion front of the sweat sample droplet 15 moves from the inlet 262 in the direction of the arrows 16 and reaches the wick 230, the sweat sample will move into the wick and be removed from the sensing chamber 282. At a time just prior to sweat sample removal, the sweat droplet volume will be at a maximum, and humidity sensor measurements taken at this point will reflect a peak humidity value (% Hₚₑₐₖ). After sweat sample removal, humidity sensor measurements will show a decrease in humidity, to a minimum humidity value (% Hₘᵢₙ), which should approach the baseline humidity value. Over time, the humidity sensor will register a series of peak humidity values, followed by returns to substantially near the minimum. Sweat rate is determined by deriving a sweat droplet volume from the change in humidity between the peak and minimum values

$$V = \frac{{m}_{{H}_{2} O}}{( \% {H}_{P e a k} - \% {H}_{Min )}} ,$$

and then dividing by the time required to reach peak humidity from the previous minimum value. Alternatively, a standard droplet volume can be determined or assumed based on factors such as inlet geometry and the surface tension of sweat. This standard droplet volume may then be used with the timing of peak and minimum humidity measurements to determine a sweat rate. To discern peak and minimum humidity values, the humidity sensor measurements must be taken with adequate frequency. For example, assume a maximum sweat rate, e.g., 500 nL/min for a 1 cm² collection area, and a 10 μL droplet size. At this rate, the droplet would reach maximum size prior to being wicked away, and the humidity sensor would measure a peak value, every 20 minutes. Therefore, humidity measurements would need to occur at least every 10 minutes. Sampling rates may be optimized to provide sufficient resolution while optimizing storage, computing and power resources on the device.

Some embodiments may include one or more temperature sensors as secondary sensors. Such temperature sensors may be configured to measure an ambient temperature, a skin temperature, or a device internal temperature. These temperature measurements may be used to inform device humidity calculations, or alternatively, the device may use external temperature or humidity measurements.

To move from the measured sweat rate to a total body sweat rate, or a sweat rate per gland, the device accounts for the known sampling area under the sweat collector. This area represents a known proportion of body surface area, and contains an approximate number of sweat glands. Total body sweat rates and per-gland sweat rates can be determined or refined in a number of ways, including accounting for generalized characteristics, such as average sweat gland density of the device mounting location, the individual's body mass index, the individual's gender, or other factors. Alternatively, the device could account for specific characteristics of the individual based on a user profile, which may include actual sweat gland density, more precise measurements of body surface area, or the use of data collected over time on the individual's sweat rate characteristics.

With further reference to FIG. 2, two features of the disclosed device, namely the wick 230 and the pump 236, provide chronologically-assured measurement accuracy, and extend the device lifespan. After a sweat sample has entered the sensing area 282, and interacts with the humidity sensor 220, it is pulled away from the humidity sensor in the direction of the arrows 16 by the wick 230. This prevents multiple sweat samples from accumulating in the sensing chamber 282, and allows new sweat samples to enter the device and be measured. The pump 236 also continuously removes moisture from the humidity dissipation volume 284, preventing the accumulation of moisture in the wick, which eventually would prevent the removal of sweat from the sensing area 282.

The pump humidity sensor 222 monitors the humidity in the pump 236, and will report to the device user in the event that the pump becomes saturated with sweat. At the point of saturation of the pump, the useable lifespan of the device is complete. The pump humidity sensor 222 may also be used to inform chronologically assured sweat sampling rates. For example, as the humidity increases in the pump 236, pump humidity will gradually converge with humidity measurements from the main humidity sensor 220. As this process occurs, the chronologically assured sampling interval will increase, meaning that as the device loses capability to remove old sweat from the wick, the device will be able to take fewer readings that measure new sweat entering the device.

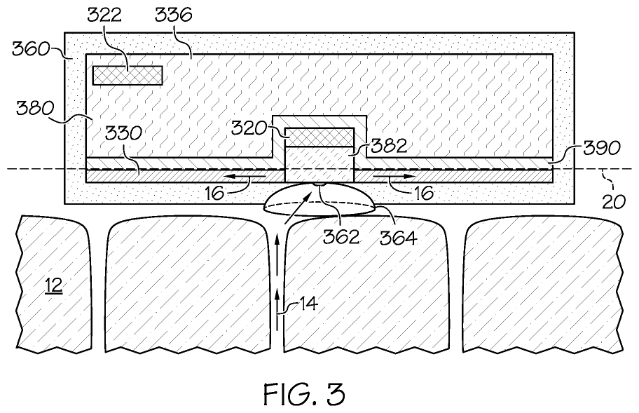

FIG. 3 represents an alternative embodiment of the disclosed invention that excludes a humidity dissipation volume, i.e., the dissipation volume is effectively zero. In this embodiment, the membrane 390 is in fluidic communication with the wick 330, and the pump 336 substantially occupies the volume occupied by the humidity dissipation volume in previous embodiments. Alternately, the enclosure volume 380 may be decreased to provide a lower profile device.

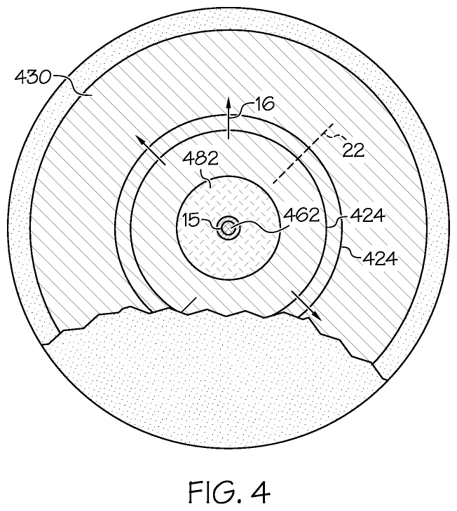

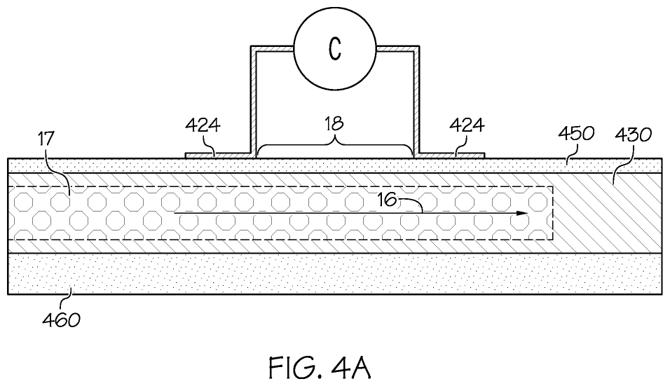

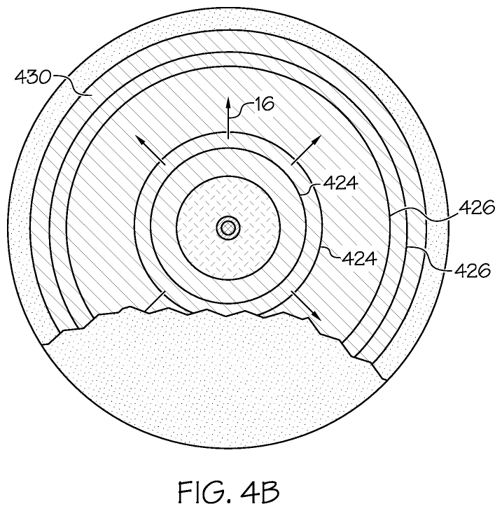

With reference to FIG. 4, where components are designated in a similar manner to like components in previous figures, an alternative embodiment of the disclosed invention is depicted. As with FIG. 2A, FIG. 4 represents a top-down view of the devices depicted in FIG. 2 and FIG. 3 as if sliced along the dashed line 20. This embodiment features an additional capacitive sensor 424 that is configured to detect changes in capacitance as a sweat sample is transported through the wick 430. As a sweat sample droplet 15 enters the device and forms at the inlet 462, it will gradually fill the sensing chamber 482 until contacting the wick 430 and moving out radially as shown by the arrows 16. The capacitive sensor 424 comprises a pair of electrodes, e.g., wires or conductive traces, configured in concentric circles and spaced a distance from the inlet 462. The electrodes are placed on top of the wick 430 and are separated from the wick by an insulation layer (not shown). With reference to FIG. 4A, which depicts a partial cross section of the device of FIG. 4 as if sliced along the dashed line 22, the capacitive sensor 424 is further illustrated. The capacitive sensor 424 is depicted with the electrodes layered on top of the insulator 450, which in turn is layered on top of the wick 430. The insulator 450 may be any suitable material that is sufficiently thin to promote device wearability, while providing sufficient electrical insulation between the wick and electrodes to promote a useful capacitance measurement, e.g., a polymer, a dielectric material, polyamide, polyester, woven, or non-woven materials. Further, the thickness of the wick also affects the required thickness of the insulator and the spacing of the electrodes. For example, the difference in dielectric constant of the wick when saturated with sweat versus the dielectric constant of the wick when dry will affect the wick thickness and/or the spacing of the electrodes. Therefore, wick thickness, insulator thickness, or electrode spacing may be adjusted separately or in combination to optimize capacitive sensor performance, and or overall device performance.

In various different embodiments, the insulator 450 may be configured to partially or completely cover the wick 430. The insulator 450 may also function as a vapor barrier, or may also function as or in place of the membrane 390 of FIG. 3. As a sweat sample 17 moves through the wick in the direction of the arrow 16, it will pass beneath the electrodes of the capacitive sensor, altering a capacitance measurement taken across the space 18 between the electrodes. The capacitance measurement may be taken by means known in the art, and may benefit from known techniques to reduce electrical noise and motion artifacts, e.g., by energizing the capacitance circuit with a sampling frequency, adding electrical shielding layers above and/or below the electrodes (not shown), or by other suitable means.

Multiple configurations of the device with capacitive sensors are possible and contemplated, and provide several advantages to the operation of the disclosed invention. For example, when used in conjunction with the humidity sensor, the capacitive sensor as depicted in FIG. 4 provides a secondary measurement of sweat rate. With the concentric electrodes of the capacitive sensor 424 spaced relatively closely to the inlet 462, the capacitive sensor provides a measurement of the frequency with which a sweat sample moves out of the sensing chamber 482 and into the wick 430. The timing of capacitance change can be compared to the humidity measurement to confirm that a droplet of sweat has exited the sensing chamber. In other embodiments, multiple capacitive sensors may be used to facilitate different functions. With reference to FIG. 4B, an embodiment of the current invention is depicted with two sets of capacitive electrode sensors 424, 426. The first capacitive sensor 424 functions as previously described to supplement the humidity sensor readings. The second capacitive sensor 426 functions as a lifetime sensor for the device. Once sweat fills the wick out to the second capacitive sensor 426, it will be saturated, and no longer capable of moving sweat away from the sensing chamber. This may signal the end of the useful life of the device, or may indicate a temporary saturation condition that can be remedied by moving moisture into the wick. Device readings taken during such temporary saturation conditions may be discarded or weighted less, or measurements may be delayed until the second capacitive sensor indicates no sweat is present.

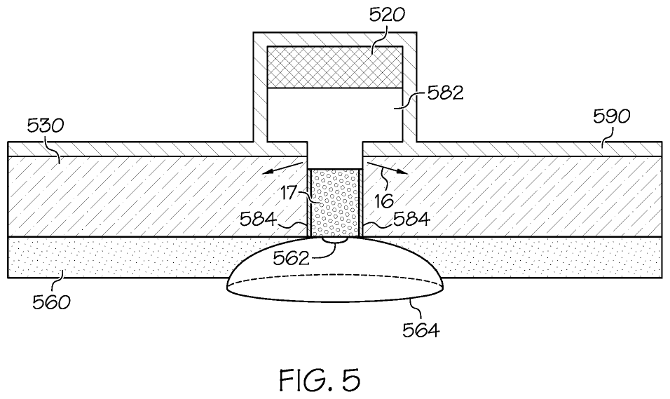

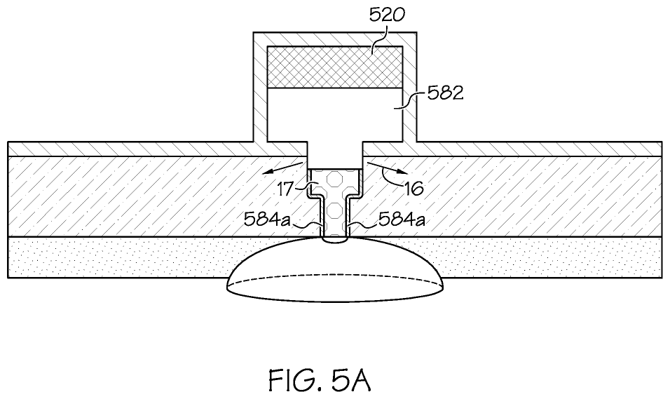

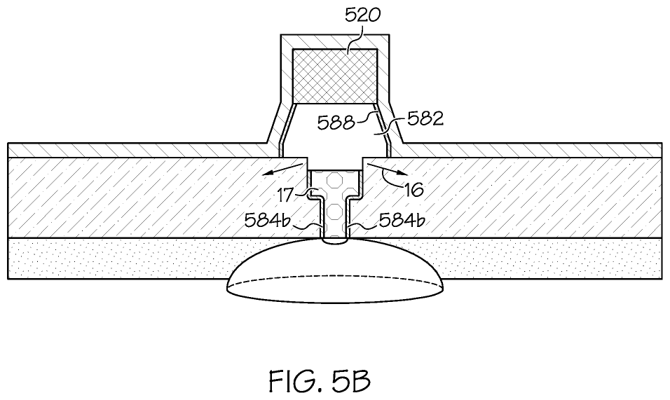

With reference to FIG. 5, where components are designated in a similar manner to like components in previous figures, another embodiment of the disclosed invention is depicted. In this and in similar embodiments, the sensing chamber 582 is shaped to reduce the potential for motion to disrupt the formation of a spherical sweat sample droplet. When placed on a wearer's skin (not shown) a sweat sample will be drawn from within the collector 564, and will enter the device via an inlet 562. As the sweat sample 17 fills into the sensing chamber 582, it fills a known sampling volume that is shaped, e.g., like a cylinder centered on the inlet. The walls of said volume include a fluid impermeable membrane or hydrophobic coating 584. The hydrophobic coating may be deposited on the wick or placed between the wick and the sampling volume. The hydrophobic coating would increase flow resistance so that sufficient pressure and or sweat volume is required to move sweat into the device. When the sweat sample volume exceeds the top of the coating 584, it will contact the wick 530 and be pulled out of the sensing chamber 582 radially in the direction of the arrows 16. When measured over time by the humidity sensor 520, the percent humidity will increase to a peak humidity value as the volume fills, and then will quickly decrease to substantially zero when the sweat sample is wicked away. The time required to fill and empty the cylindrical volume, coupled with the volume of sweat in the cylindrical volume provides a sweat rate. While the invention is depicted without a humidity dissipation volume or a pump, such components may be combined with the present embodiments.

With reference to FIG. 5A, another embodiment of the device of FIG. 5 is depicted which has a decreased sweat sample volume, while maintaining a significant humidity change that facilitates accurate sensing. Having a lower sample volume will allow the device to take more frequent measurements, since the sampling volume will fill and empty more rapidly. A lower volume may also be more consistently pulled into the wick, meaning the entire volume is more frequently wicked away, again improving device accuracy. Requiring less sweat also improves device function at lower sweat rates. In this embodiment, the sensing chamber 582 includes a sampling volume having a first portion located nearest the inlet and a second portion located nearest the humidity sensor. The first portion is narrower than the second portion, i.e., it has a smaller volume than the second portion. In operation, a sweat sample droplet 17 fills the narrow first portion of the sampling volume, and then enters the second portion, which has a greater surface area. Once the sweat sample reaches the top of the coating 584a, it contacts the wick and substantially all of the sample is moved away radially as depicted by the arrows 16. The humidity sensor will measure a substantial drop in humidity, while requiring a lower volume to be filled with sweat.

With reference to FIG. 5B, another embodiment of the device of FIG. 5 is depicted. In this embodiment, the sensing chamber 582 is shaped to facilitate removal of water vapor that accumulates in the sensing chamber. The sensing chamber 582 wall intersects the wick 530 so that any vapor that condenses on the wall moves onto the wick and is transported radially in the direction of the arrows 16. An optional hydrophobic coating 588 may cover the inner surface of the wall, aiding the movement of condensed vapor toward the wick. Any of the sensing chamber embodiments of FIGS. 5-5B may be practiced with the embodiments depicted in the previous figures.

The above-described configurations represent a basic foundation for either a simple device or a more complex device. Some embodiments of the disclosed invention may therefore include additional materials, components, designs, or other features for operation, as long as the device uses at least one humidity sensor to measure sweat rate.

This has been a description of the present invention along with a preferred method of practicing the present invention, however the invention itself should only be defined by the appended claims.

1. A device, comprising:
a fluid-impermeable substrate for defining a device volume and interacting with a device wearer's skin;
a sweat collector;
a sensing chamber in fluidic communication with the sweat collector through an inlet, wherein the sensing chamber comprises a humidity sensor, a partially sealed space having a volume, and a droplet formation area surrounding the inlet, wherein the droplet formation area has a hydrophobic coating;
a wick in fluidic communication with the sensing chamber;
a humidity dissipation volume in fluidic communication with the wick; and
a pump comprising a membrane, and an absorbent material, and wherein the membrane has a first side adjacent to the humidity dissipation volume and a second side adjacent to the absorbent material

2. The device of claim 1, further comprising a pump humidity sensor that is in fluidic communication with the absorbent material.

3. The device of claim 1, wherein the humidity dissipation volume has a volume of zero.

4. The device of claim 1, further comprising one or more secondary sensors, wherein the one or more secondary sensors is chosen from the following: a sensor for measuring an ambient temperature, a sensor for measuring a skin temperature, a sensor for measuring a device internal temperature, a volumetric sweat rate sensor, a micro-thermal flow rate sensor, a discrete volume dosing system sweat rate sensor, a galvanic skin response sensor, a sweat conductivity sensor, an impedance skin contact sensor, and a capacitive sensor.

5. The device of claim 1, further comprising:
a first capacitive sensor, comprising an insulator at least partially covering the wick, wherein the insulator has a first side that is adjacent to the wick and a second side; two conductive traces arranged in concentric circles centered on the sensing chamber and situated on the second side of the insulator, and electronics for measuring a capacitance across the conductive traces.

6. The device of claim 5, further comprising:
a second capacitive sensor, comprising two conductive traces arranged in concentric circles centered on the sensing chamber and situated on the second side of the insulator, and electronics for measuring a capacitance across the conductive traces, wherein the first capacitive sensor is located between the second capacitive sensor and the sensing chamber.

7. The device of claim 5, wherein the insulator and the membrane are the same component.

8. A device, comprising:
a fluid-impermeable substrate for defining a device volume and interacting with a device wearer's skin;
a sweat collector;
a sensing chamber in fluidic communication with the sweat collector through an inlet, wherein the sensing chamber comprises a humidity sensor, a partially sealed space having a chamber volume, and a hydrophobic coating defining a sample volume;
a wick in fluidic communication with the sensing chamber and partially separated from the sample volume by the hydrophobic coating;
a humidity dissipation volume in fluidic communication with the wick; and
a pump comprising a membrane, and an absorbent material, and wherein the membrane has a first side adjacent to the wick and a second side adjacent to the absorbent material.

9. The device of claim 8, further comprising a pump humidity sensor that is in fluidic communication with the absorbent material.

10. The device of claim 8, wherein the humidity dissipation volume has a volume of zero.

11. The device of claim 8, further comprising one or more secondary sensors, wherein the one or more secondary sensors is chosen from the following: a sensor for measuring an ambient temperature, a sensor for measuring a skin temperature, a sensor for measuring a device internal temperature, a volumetric sweat rate sensor, a micro-thermal flow rate sensor, a discrete volume dosing system sweat rate sensor, a galvanic skin response sensor, a sweat conductivity sensor, an impedance skin contact sensor, and a capacitive sensor.

12. The device of claim 8, further comprising:
a first capacitive sensor, comprising an insulator at least partially covering the wick, wherein the insulator has a first side that is adjacent to the wick and a second side; two conductive traces arranged in concentric circles centered on the sensing chamber and situated on the second side of the insulator, and electronics for measuring a capacitance across the conductive traces.

13. The device of claim 12, further comprising:
a second capacitive sensor, comprising two conductive traces arranged in concentric circles centered on the sensing chamber and situated on the second side of the insulator, and electronics for measuring a capacitance across the conductive traces, wherein the first capacitive sensor is located between the second capacitive sensor and the sensing chamber.

14. The device of claim 8, wherein the sample volume has one of the following shapes: a cylinder, and a shape comprised of a first cylinder having a first radius, and a second cylinder having a second radius, wherein the second radius is larger than the first radius, and the first cylinder is between the inlet and the second cylinder.

15. The device of claim 8, wherein the sensing chamber further comprises a containment wall having an internal surface, a hydrophobic coating on the internal surface, and wherein the containment wall is shaped to facilitate the movement of condensed vapor to the wick.

16. A method of measuring a sweat rate, comprising:
receiving a plurality of sweat samples in a sensing chamber, wherein the sensing chamber has a chamber volume and each of the plurality of sweat samples has a sample volume;
using a sensor to take a plurality of humidity measurements of the chamber volume;
determining a maximum humidity measurement and a baseline humidity measurement;
correlating the baseline humidity measurement to an empty time when a sweat sample is not in the chamber, and correlating the maximum humidity measurement to a filled time when a sweat sample is in the sensing chamber;
determining a fill duration, as a difference between the empty time and the filled time; and
calculating a sweat rate from the fill duration and the sample volume.

17. The method of claim 16, further comprising:
using a first capacitance sensor to measure a first wetting time in a wick, wherein the first wetting time is when a sweat sample is near enough to interact with the first capacitance sensor;
comparing the first wetting time to the empty time; and
determining that a sweat sample has exited the sensing chamber and entered the wick.

18. The method of claim 17, further comprising:
using a second capacitance sensor located between the first capacitance sensor and an outer edge of the wick to measure a second wetting time in the wick, wherein the second wetting time is when a sweat sample is near enough to interact with the second capacitance sensor; and determining that a device lifespan has elapsed as of the second wetting time.
